(19) United States
(12) Patent Application Publication
(10) Pub. No.: US 2026/0115472 A1
(43) Pub. Date: Apr. 30, 2026

(54) TINNITUS SUPPRESSION DEVICE, SYSTEM AND COMPUTER PROGRAM

(71) Applicant: CereGate GmbH, Munich (DE)

(72) Inventors: Saman HAGH GOOIE, Munich (DE); Bálint VÁRKUTI, Munich (DE); Ricardo SMITS SERENA, Munich (DE)

(21) Appl. No.: 19/116,802

(22) PCT Filed: Sep. 30, 2022
(86) PCT No.: PCT/EP2022/077377

§ 371 Date: Mar. 28, 2025

(51) Int. Cl. A61N 1/36; A61N 1/372; G10L 25/51; H04R 25/00

(52) U.S. Cl. CPC A61N 1/361; A61N 1/36057; A61N 1/36139; A61N 1/37241; A61N 1/37247; G10L 25/51; H04R 25/75; H04R 2225/67

(57) ABSTRACT

The present disclosure relates to a tinnitus suppression device, method, system and computer program. In some aspects, a tinnitus suppression device comprises a receiver module configured to receive sound signals, a processing module operably connected to a memory and to the receiver module, and a neurostimulation module operably connected to the processing module. In some aspects, the processing module and the neurostimulation module may be configured to determine a received sound signal that corresponds to a template stored in the memory, that is above a preconfigured threshold, preferably above a preconfigured threshold which is specific for the individual, or both. A multi-channel neurostimulation signal encoding the determined sound signal may be generated and applied to a neurostimulation device of the individual that may be configured to directly stimulate afferent sensory neurons of the central nervous system of the individual and thereby to elicit, for each channel of the neurostimulation signal, one or more non-auditory, preferably somatosensory, perceptions in a cortex area of the individual.

### 1. TECHNICAL FIELD

The present disclosure relates to a tinnitus suppression device, system, method and computer program configured for suppressing a tinnitus perception.

### 2. TECHNICAL BACKGROUND

Tinnitus is the perception of sound when no corresponding external sound is present. While often described as a ringing, it may also sound like a clicking, buzzing, hiss, or roaring. The sound may be soft or loud, low or high pitched, and often appears to be coming from one or both ears or from the head itself. In some people, the sound may interfere with concentration and in some cases, it is associated with anxiety and depression. Tinnitus is usually associated with a degree of hearing loss and with decreased comprehension of speech in noisy environments. It is common, affecting about 10-15% of people.

Tinnitus may result from various underlying causes and may be generated at any level of the auditory system and structures beyond that system. The most common causes are hearing damage, noise-induced hearing loss or age-related hearing loss, known as presbycusis; and tinnitus can suddenly emerge during a period of emotional stress.

U.S. Pat. No. 10,198,076 B2 relates to a method for providing information to a user, the method including: receiving an input signal from a sensing device associated with a sensory modality of the user, generating a preprocessed signal upon preprocessing the input signal with a set of preprocessing operations; extracting a set of features from the preprocessed signal, processing the set of features with an artificial neural network system, mapping outputs of the neural network system to a device domain associated with a device including a distribution of haptic actuators in proximity to the user, and at the distribution of haptic actuators, cooperatively producing a haptic output representative of at least a portion of the input signal, thereby providing information to the user.

US 2021/0325969 A1 relates to a method for haptic stimulation that includes: receiving an audio input, determining a set of parameters based on the audio input, determining a set of stimulation locations based on a collective set of energy parameters, assigning a vibration intensity to a set of one or more haptic actuators, and stimulating a user at the set of haptic locations based on the vibration intensities.

US 2022/0126094 A1 relates to a method for multimodal stimulation that functions to provide therapy to a user for tinnitus or other conditions, and includes: receiving a set of inputs, determining a set of outputs, providing the set of outputs to a user, and adjusting any or all of the set of outputs.

U.S. Pat. Nos. 9,786,201 B2 and 9,679,546 B2 both relate to vibratory motors that are used to generate a haptic language for music or other sound that is integrated into wearable technology.

EP 3 574 951 B1 relates to an apparatus and method for use in treating tinnitus, which employs a sound processing unit, a tactile unit, and an interface therebetween. The tactile unit comprises an array of stimulators each of which can be independently actuated to apply a tactile stimulus to a subject, and the tactile unit comprises an input for receiving a plurality of actuation signals from the interface and directing individual actuation signals to individual stimulators.

U.S. Pat. No. 9,078,065 B2 relates to a method and a system for presenting audio signals as vibrotactile stimuli to the body in accordance with a Model Human Cochlea. Audio signals are obtained for presentation. The audio signals are separated into multiple bands of discrete frequency ranges that encompass the complete audio signal. Those signals are output to multiple vibrotactile devices. The vibrotactile devices may be positioned in a respective housing to intensify and constrain a vibrational energy from the vibrotactile devices.

Applicant's own U.S. Pat. No. 11,344,725 B2 relates to a system for providing neural stimulation signals. The system is configured to elicit sensory percepts in the cortex of an individual that may be used for communicating conceptual information to an individual. The system comprises means for selecting at least one neural stimulation signal to be applied to at least one afferent axon directed to at least one sensory neuron in the cortex of the individual. The at least one neural stimulation signal corresponds to the conceptual information to be communicated. The system further comprises means for transmitting the at least one neural stimulation signal to stimulation means of the individual.

US 2016/0012688 A1 relates to providing information to a user through somatosensory feedback. A hearing device is provided to enable hearing-to-touch sensory substitution as a therapeutic approach to deafness. By way of signal processing on received signals, the hearing device may provide better accuracy with the hearing-to-touch sensory substitution. For example, the tactile interface devices may be vibrating devices attached to a vest, which is worn by the user.

U.S. Pat. No. 8,065,013 B2 relates to a method of transitioning stimulation energy (e.g., electrical stimulation pulses) between a plurality of electrodes that are implanted within a patient.

U.S. Pat. No. 10,437,335 B2 relates to a wearable Haptic Human/Machine Interface (HHMI) which receives electrical activity from muscles and nerves of a user. An electrical signal is determined having characteristics based on the received electrical activity. The electrical signal is generated and applied to an object to cause an action dependent on the received electrical activity. The object can be a biological component of the user, such as a muscle, another user, or a remotely located machine such as a drone.

U.S. Pat. No. 10,869,142 B2 relates to a new binaural hearing aid system, which is provided with a hearing aid in which signals that are received from external devices, are filtered with binaural filters in such a way that a user perceives the signals to be emitted by respective sound sources positioned in different spatial positions in the sound environment of the user, whereby improved spatial separation of the different sound sources is facilitated.

### 3. SUMMARY OF THE INVENTION

As explained above, several attempts have been made in the prior art to provide for improved hearing aids and/or for treatment or amelioration of tinnitus, e.g., via tactile stimulators. However, the prior art methods, devices and systems have various deficiencies. As discussed above, the methods, devices and systems known from the prior art may, for example, not be suited to treat or ameliorate tinnitus in an easy and flexible manner or may not fully achieve sufficient tinnitus suppression. Further, some prior art solutions partially rely on complex devices such as wearables which are bulky and cumbersome and/or might interfere in an unnatural way with the normal behavior of an individual. It is thus a problem underlying the present invention to overcome such and similar deficiencies of previous technologies.

This and similar problems are at least partially solved by the tinnitus suppression device, system and computer program specified in the appended claims. The provided tinnitus suppression device, system and computer program allow to suppress and/or treat tinnitus in a flexible and efficient manner that cannot be achieved in the same way with prior art technologies.

An aspect of the present disclosure provides a tinnitus suppression device for an individual, comprising: a receiver module (or receiver) configured to receive sound signals (e.g., analog or digital electrical signals generated by a microphone or obtained from remote sound transducer apparatus), a processing module (or processor) operably connected to a memory and to the receiver module, and a neurostimulation module (or stimulator operably connected to the processing module, wherein the processing module and the neurostimulation module are configured to: determine a received sound signal that corresponds to a template stored in the memory, that is above a preconfigured threshold, preferably above a preconfigured threshold which is specific for the individual, or both, and generate a multi-channel neurostimulation signal encoding the determined sound signal, and apply the generated multi-channel neurostimulation signal to a neurostimulation device (e.g., a multi-channel neurostimulation electrode) of the individual configured to directly stimulate afferent sensory neurons of the central nervous system, CNS (i.e., of the brain and/or the spinal cord), of the individual and thereby to elicit, for each channel of the neurostimulation signal, one or more non-auditory, preferably somatosensory, perceptions in a cortex area (e.g., a somatosensory cortex area) of the individual. In some aspects, the processing module may be configured such, that the non-auditory perception elicited by the multi-channel neurostimulation signal is perceived by the individual substantially simultaneously with a corresponding physiologically normal auditory perception.

For example, the preconfigured threshold may be an auditory perception threshold of the individual that, in some aspects, may be related to a specific frequency band or a template from a preconfigured sound library. For example, the preconfigured threshold may correspond to a hearing threshold specific for the individual or to an absolute threshold of hearing (ATH) which typically is defined as the minimum sound level of a pure tone that an average human ear with normal hearing can hear with no other sound present. It should be noted that the ATH is not a discrete point, and is therefore classed as the point at which a sound elicits a response a specified percentage of the time. This is also known as the auditory threshold. The threshold of hearing is generally reported as the RMS sound pressure of 20 micropascals, i.e., 0 dB SPL. However, the preconfigured threshold could also comprise an offset with respect to the ATH or to hearing threshold specific for the individual to ensure that only strong enough real sounds are transformed to a non-auditory perception for tinnitus suppression. For example, such a hearing threshold specific to the individual may be obtained via psycho-acoustic testing as known in the art.

Further, the preconfigured threshold may specifically be sensitive to a single or multiple frequency ranges and/or be specifically sensitive to a sound from a stored sound library. For example, if the tinnitus perception is typically triggered by certain external sounds such as a sound of an alarm clock, then the processing module can be configured to detect this sound via a microphone and then activate appropriate perceptual channels to remove the individual's sensitivity.

As discussed in more detail with reference to FIG. 9 below, aspects of the present disclosure thus allow to establish an independent, non-auditory sense of hearing that is not affected by tinnitus perceptions/sensations and thus provides the brain with a means for discriminating which auditory sound perceptions are real and which are hallucinatory/tinnitus related. In this way, aspects of the present disclosure enable the brain to learn to distinguish between real and hallucinatory/tinnitus related perceptions/sensations and to suppress the later based on the learning.

In some aspects, the memory/data storage module may stores parameters characterizing a tinnitus perception of the individual and the processing module and the neurostimulation module may be further configured to compare the received sound signal to the stored parameters characterizing a tinnitus perception of the individual, to determine, based on the comparison, whether an auditory perception of the received sound signal is distinguishable from the tinnitus perception of the individual, and signaling to the individual, via the neurostimulation device, whether the multi-channel neurostimulation signal corresponds to a sound signal that is distinguishable from the tinnitus perception of the individual or not. For instance, such signaling may be performed using an auxiliary perceptual channel that may be established as described in applicant's own U.S. Pat. No. 11,344,725 B2.

In this manner, the tinnitus suppression performance can be further enhanced due to supervised learning as real sounds that are indistinguishable from tinnitus perceptions can be flagged. Further, the stored parameters may be associated with one or more of: an auditory perception threshold related to a specific frequency band or a sound template from a preconfigured sound library. For instance, a library of various tinnitus perceptions typically perceived by an individual may be generated via psycho-acoustic testing and may be used for deriving the stored parameters.

In some aspects, the tinnitus suppression device as disclosed herein may further comprise a user input interface operably connected to the processing module and configured to receive a user input indicating a tinnitus state of the individual (e.g., whether a tinnitus perception is present or not, which type of tinnitus perception is present, etc.) wherein, in response to the user input indicting the tinnitus state of the individual, the processing module and the neurostimulation module are further configured to: generate a multi-channel neurostimulation signal encoding a non-auditory perception configured to suppress the tinnitus state of the individual and apply the generated multi-channel neurostimulation signal to a neurostimulation device of the individual.

Further, in some aspects, the processing module and the neurostimulation module may be further configured to determine that a received sound level is, for a preconfigured duration, below a second preconfigured threshold, preferably below a second preconfigured threshold that is specific for the individual, and generate, based on the determination, and optionally based on stored parameters characterizing a tinnitus perception of the individual, a multi-channel neurostimulation signal encoding a non-auditory perception configured to suppress a tinnitus state of the individual and apply the generated multi-channel neurostimulation signal to a neurostimulation device of the individual.

For example, such a multi-channel neurostimulation signal that encodes the non-auditory perception configured to suppress the tinnitus state of the individual may comprises a multi-channel neurostimulation signal encoding a non-auditory noise signal. For example, such a noise signal may have a power spectrum corresponding to white noise, pink noise, grey noise, blue noise, violet noise or Brownian noise. In this manner, the tinnitus suppression device may not only enable the brain to learn how to suppress tinnitus perceptions/sensations but may also help to reduce tinnitus perceptions/sensations by providing non-auditory background noise that the brain can interpret as non-silence.

Further, the processing module and the neurostimulation module may further be configured to randomly cycle between different multi-channel neurostimulation signals each encoding a different noise signal. Alternatively, or additionally, the processing module and the neurostimulation module may further be configured to generate bursts of the multi-channel neurostimulation signal encoding a specific noise signal such that there are rhythmic active and silent segments delivered to the individual. In this manner cortical adaptation processes that may reduce tinnitus suppression performance may be circumvented or at least reduced.

Further, in some aspects, encoding by the processing module may comprise applying a filter operation to the received sound signal to generate a plurality of subcomponent signals of the sound signal and mapping each subcomponent signal to a different channel of the multi-channel neurostimulation signal. For instance, the sound signal can be decomposed with a method that is chosen on the basis of how much information the neural interface can transmit.

Further, said filter operation may involve performing spectral analysis, wavelet analysis, principal component analysis, independent component analysis, using a filter bank, and/or a combination thereof. In a simple example, as illustrated in FIG. 3 in section 4. below, a received sound signal (e.g., a sample of speech or a sample of a piece of music, etc.) may be subdivided (e.g., via a bank of N bandpass filters) into N subcomponent signals corresponding to N different frequency bands.

Via encoding sound signals in multiple, non-auditory perceptual channels the tinnitus suppression device can enable or support sound perception even for patients that cannot be treated via conventional techniques. Moreover, not being limited to the physiologic structure and function of the auditory nerve and upstream auditory processing may substantially improve flexibility, channel count and the fidelity of sound signal representation. In this manner, even complex auditory stimuli such as speech in a cocktail party environment or classical music can be perceived with sufficient fidelity.

In the same manner as an infant's brain is capable of associating syntactic meaning with perceived auditory stimuli through (repetitive) interaction with the physical/auditory environment (e.g., via reinforcement learning), a patient can learn to associate the information content of physical sound signals (e.g., the conceptual information encoded in speech, traffic noise, music, etc.) with the non-auditory perceptions elicited by the multi-channel neurostimulation signal. In order to do so, it is important that the neural representation of the physical sound signal that is generated by the multi-channel neurostimulation signal is complex and variable enough that the relevant information content can be preserved during auditory processing and subsequent neurostimulation.

In some embodiments, the processing module may be configured to determine, preferably via an on-line auto-calibration procedure, a maximal number N of different perceivable perceptual channels that are specific for the individual and select the applied filter operation based on the determination, such that a fidelity of a representation of the received sound signal by the plurality of subcomponent signals is maximized for the determined number of perceptual channels. For example, after the maximal number N of usable perceptual channels is determined, independent component analysis or a similar filter operation can be applied to the received sound signal in order to subdivide it into N subcomponent signals in such a manner that the information content/entropy of the neural representation of the sound signal elicited by applying the subcomponent signals to the afferent neurons is maximized.

Such an on-line autocalibration of the neural interface device/neurostimulation signal may be based on observing the excitation behavior or neural activation function of afferent sensory nerve fibers that can be stimulated by a given neurostimulation means such as a SCS-electrode or DBS electrode connected to corresponding a neurostimulation module or device. This approach is based on the insight that there exist strong correlations between the highly non-linear bioelectric response of an active stimulated afferent sensory nerve fiber (e.g., ECAP) or plurality of such fibers and a corresponding artificial sensory perception/artificial sensation elicited in a sensory cortex area of the individual. This non-linear bioelectric response essentially serves as a fingerprint of the afferent sensory nerve fiber that can be measured and used for on-line recalibration of neurostimulation signal parameters for direct neurostimulation of afferent sensory neurons targeting directly or indirectly (i.e., via multi-synaptic afferent pathways) sensory neurons in a specific target sensory cortex area. In this manner, long-term stability of highly specific, fine-grained and multi-dimensional information transfer to the brain can be ensured.

More specifically, the tinnitus suppression device may be configured (e.g., via a suitable firmware routine or software application) to carry out an on-line auto-calibration procedure that may comprise the following steps: determining a plurality of independently operable stimulation electrodes or contacts of a neurostimulation interface operably connected to or integrated with the neural interface device; choosing a set of test signal parameters preferably associated with a set of N output qualities of a sound processor; generating, based on the chosen set of test signal parameters, a plurality of neurostimulation test signals configured to elicit a bioelectric response in one or more afferent sensory neurons of the individual; applying the generated plurality of neurostimulation test signals to the afferent sensory neurons via one or more of the determined plurality of stimulation electrodes or contacts of the neurostimulation interface; sensing, via the neurostimulation interface, one or more bioelectric responses of the one or more stimulated afferent sensory nerve fibers; and determining, based on the sensed bioelectric responses, a number N of different sensations that can independently be elicited in one or more cortex areas of the individual via neurostimulation of the one or more afferent sensory nerve fibers.

For instance, determining the N different (artificial) sensations may comprises comparing the sensed bioelectric responses with a set of reference responses stored in a memory module of the neural interface device or obtained via a wired or wireless communication interface of the neural interface device.

Further, determining, for one or more of the N determined sensations and based at least partially on the sensed bioelectric responses, a dynamic range of one or more neurostimulation signals that are configured to elicit the one or more determined sensations; and, optionally, subdividing the determined dynamic range into M, preferably equidistant, intervals. In this manner, the symbol count (e.g., S0=low intensity, S1=medium intensity, S2=high intensity) of each perceptual channel can be determined and optimized to maximize channel capacity.

The auto-calibration procedure may further comprise receiving, via a communication interface or user interface of the neural interface device, sensory feedback information from the individual associated with one or more of the sensations elicited by the plurality of neurostimulation test signals; and using the sensory feedback information for determining and/or characterizing the N different sensations and/or using the sensory feedback information for determining and/or subdividing the determined dynamic range of the one or more neurostimulation signals that are configured to elicit the one or more determined sensations.

In this manner, the fidelity of perceptual channel characterization can be improved, since the recorded bioelectric responses can be correlated with the (subjective) sensory feedback information provided by the patient/individual. For instance, the feedback information may comprise one or more indications of one or more of the following characteristics of the elicited sensations: a sensory modality, a location, an intensity and a frequency.

Determining the number N of usable perceptual channels (and the number M of symbols/differentiable perceptual levels/qualities per channel) in this manner allows the filters/signal transformations to be applied in a dynamic manner to the received sound signal, so that the fidelity of the neural representation is adapted (e.g., maximized) in real-time and in an on-line fashion in sync with the auto-calibration. For instance, if the relative distance between the stimulation electrode and the targeted afferent sensory neurons changes (e.g., due to a slow drift of a SCS-electrode or due to a movement of the patient), stimulation parameters can be adjusted such that the number of distinct perceptual channels and thereby sound signal representation fidelity stays as large as possible.

In some embodiments, the processing module may be further configured to apply the filter operation according to multiple selectable filter modes wherein the generation of the subcomponent signals and/or the mapping of the subcomponent signals to the multiple channels of the neurostimulation signal may be based on the selected filter mode. For instance, the filter mode may be user selectable (e.g., via a user interface) or automatically determined by the processing module.

For instance, the processing module may be further configured to determine, preferably based on an analysis of the received sound signal, an auditory environment and/or a likely type of sound signal source associated with the received sound signal; and encode the received sound signal based on the determined auditory environment and/or type of sound signal source. This allows the tinnitus suppression device to maximize, for a given number of perceptual channels and a likely sound signal source or auditory environment the information content the neural representation of the received sound signal contains.

For instance, certain frequency bands, phoneme subcomponent signals, musical instrument subcomponent signals or more abstract subcomponents signals may, for a whole class or subclass of received sound signals (e.g., speech, classical music), typically contain the majority of the information content of the received sound signal whereas other frequency bands/subcomponent signals mainly contain noise. Thus, by determining the auditory environment and/or the likely type of sound signal source, the processing module can select a filter operation best suited for an expected class sub-class of sound signals. For instance, the processing module may select a set of Gabor filters forming a Gabor filter bank best suited for extracting the spectro-temporal information that is typical for speech signals whereas a band pass filter bank with adjustable gains and bandwidths may be better suited for perceiving an orchestra playing classical music.

Moreover, also the set of perceptual channels may be adjusted based on the determined auditory environment and/or a likely type of sound signal source. For instance, a set of distinct somatosensory sensations (e.g., a subset of the dermatomes or peripheral nerve fields of the back side of the torso; see FIG. 1 below) might be best suited for perceiving classical music and experiencing the joy in doing so whereas a set of phosphenes, e.g., perceived in the periphery of the retina may be best suited for speech perception, e.g., via mapping a set of Gabor-filtered subcomponent signals to a set of phosphenes that can be distinguished by the individual as different vowels, consonants, phonemes etc.

In general, the multiple filter modes may comprise one or more of the following: a speech perception mode, a music perception mode, a closed space mode, an open space mode, a foreign language mode, a multi-source environment mode and a traffic mode. Additionally, or alternatively, the processing module may be configured to select the filter mode based on the determined auditory environment and/or likely type of sound signal source.

Further, for example to improve the fidelity of the neural sound signal representation, each filter mode may be associated with a plurality of filters being applied to the received sound signal to generate the plurality of subcomponent signals, wherein the filters may comprise bandpass filters, wavelet filters and/or Gabor filters or the like.

Alternatively or additionally, the filters may be configured to filter out distinct characteristics of the received sound signal that are typical for an auditory environment and/or a likely type of sound signal source associated with the selected filter mode. For example, different sets of filters/filter functions may be designed for filtering out vowels, consonants, phonemes, musical instruments, cars, animals, etc. and stored in a memory device of the tinnitus suppression device. When the processing module determines, for example, that the likely sound source is music, it might access the memory device and retrieve a set of filters designed for music perception. As discussed above, this pre-configured set may then be further adapted based on the number N of available perceptual channels. For instance, in some embodiments the number N of channels of the neurostimulation signal may be at least 2 (for representing simple sound characteristics), preferably at least 5 and more preferably at least 20 (for almost natural speech perception).

Additionally, the number of different perceivable perceptual qualities per perceptual channel (e.g., the number of different intensities that can be perceived per channel) may larger than 2 (e.g., loud vs. quiet), preferably larger than 3 (e.g., loud, medium, quiet) and more preferably larger than 10 (e.g., spanning 30 dB of sound pressure level in steps of 3 dB). As mentioned in a slightly different context above, the processing module may be configured to execute an autocalibration procedure, preferably interleaved with normal operation, to determine, for a given neurostimulation means or device of the individual, the number of differentiable perceptual channel and/or the number differentiable levels per channel.

To assist the individual's brain in perceiving sound using the tinnitus suppression device of the present disclosure, e.g., assist with extracting the information content of speech, at least one of the multiple channels of the multi-channel neurostimulation signal may be an auxiliary channel that encodes at least one of the following characteristics of the received sound signal, a sound power or amplitude, a sound pitch, a sound timing, a direction of the sound signal source and a motional state of the sound signal source. For instance, the processing module may be configured to determine the direction, distance and/or the velocity vector (i.e., direction and magnitude) of a (moving) sound signal source and encode this information in one or more of perceptual channels established by the multi-channel neurostimulation signal. For example, if two or more spatially separated sound sensors provide sound signals to the tinnitus suppression device, arrival time difference, a phase difference and/or a sound signal amplitude difference may be used to determine the spatial direction of a sound signal source. If the type of sound signal source is known, also the total distance may be determined from an amplitude comparison with a reference sound signal. Finally, by determining a Doppler shift associated with sound signals received from a moving sound signal source also the magnitude and direction (i.e. approaching or receding) of the velocity vector can be determined and subsequently communicated to the individual.

For instance, in some embodiments, the sound signal may be received from at least two spatially separated sound sensors and the processor may be configured to determine a direction of the sound signal source based on information in the sound signal associated with the at least two spatially separated sound sensors, preferably based on a phase difference, a timing difference and/or an sound signal amplitude difference associated with the spatial separation of the at least two sound sensors. Alternatively or additionally, the channel that encodes the sound signal direction may be configured to elicit somatosensory perceptions in adjacent areas of a body part, wherein each area corresponds to a different direction.

According to some embodiments, such an auxiliary channel may also encode context information associated with the received sound signal such as information about the sound signal source, a sound signal start or stop indication, one or more sign language symbols associated with the received sound signal, an indication of the emotional state of the sound signal source; and indication of the language used by the sound signal source.

For instance, if the disclosed tinnitus suppression device is operated in conjunction with DBS-equipment, the auxiliary channel may even use a different type of perception than the channels used for sound perception. For instance, in a dual-interface configuration a (multi-channel) SCS-electrode may be used by the tinnitus suppression device to elicit a plurality of sound perceptions representing the received sound signal and a DBS-electrode may be used to elicit artificial sensations/perceptions of a different type/modality, such as vision or smell to implement the auxiliary channel. For example, different taste sensations may be used to encode the emotional state of a speaker (sour=angry, sweet=kind, bitter=joyfull, etc.) thereby providing essential context that supports speech perception and extraction of syntactic meaning from the sound signal representations perceived by the individual.

Further, the neurostimulation signal may be configured such that adjacent channels of the neurostimulation signal elicit somatosensory perceptions in adjacent areas of a body part of the individual or in adjacent body parts, preferably in a tonotopic manner. In this manner, patients that were used to normal cochlear sound processing, that also is based on a tonotopic organization of the sensory cells in the cochlear, will more easily adapt to the tinnitus suppression device.

Further, the neurostimulation signal may be configured such that the areas of the body part are arranged in an essentially 2D array and, wherein one direction of the array encodes sound source direction, and the other direction is used for mapping the adjacent channels. More generally, as illustrated in FIG. 1 below different sound representation channels may be mapped to different dermatomes and/or sub-areas of a dermatome, e.g., via using a look-up table.

Some aspects may further comprise the tinnitus suppression device as discussed above and one or more sound sensors providing input signals to the receiver module, and optionally, a neurostimulation device for stimulating afferent sensory neurons in the brain and/or the spinal cord of the individual.

A further aspect of the present disclosure relates to a tinnitus suppression system, comprising the tinnitus suppression device as disclosed herein, a sound generator operably connected to a loud speaker, a memory storing a plurality of training sounds comprising a subset of sound signals that are indistinguishable from one or more tinnitus perceptions of an individual and a training module operably connected to the tinnitus suppression device, the memory and the sound generator and configured to select a set of training sounds from the stored plurality of sound signals comprising a subset of sound signals that are indistinguishable from one or more tinnitus perceptions of individual and to present the selected set of training sounds to the individual via the sound generator and the tinnitus suppression device essentially simultaneously.

In some aspects, such a tinnitus suppression system may further comprise an input interface operably connected to the memory and configured to receive parameters characterizing one or more tinnitus perceptions of the individual and the training module may be configured to generate, based on the received parameters, a first set of training sounds that are indistinguishable from one or more tinnitus perceptions of the individual and a second set of training sounds that are distinguishable from one or more tinnitus perceptions of the individual.

Such a tinnitus suppression system may be used in various tinnitus suppression training sessions to speed up learning to distinguish between real and hallucinatory/tinnitus related perceptions/sensations.

Further aspects relate to a computer program, comprising instructions for carrying out the following steps, when being executed by a tinnitus suppression device: determine a received sound signal that corresponds to a template stored in the memory, that is above a preconfigured threshold, preferably above a preconfigured threshold which is specific for the individual, or both, and generate a multi-channel neurostimulation signal encoding the determined sound signal, and apply the generated multi-channel neurostimulation signal to a neurostimulation device of the individual configured to directly stimulate afferent sensory neurons of the central nervous system, CNS, of the individual and thereby to elicit, for each channel of the neurostimulation signal, one or more non-auditory, preferably somatosensory, perceptions in a cortex area of an individual.

Such a computer program may comprise further instructions for operating the tinnitus suppression device in order to implement the functionalities as described above for the various embodiments of the tinnitus suppression device. As disclosed with reference to FIG. 12 below, a corresponding tinnitus suppression method is also part of the present disclosure.

Further aspects relate to a computer program, comprising instructions for carrying out the following steps, when being executed by a tinnitus suppression system: select a set of training sounds from a stored plurality of sound signals comprising a subset of sound signals that are indistinguishable from one or more tinnitus perceptions of an individual, and present the selected set of training sounds to the individual via a sound generator and the tinnitus suppression device essentially simultaneously.

Such a computer program may comprise further instructions for operating the tinnitus suppression device in order to implement the functionalities as described above for the various aspects of the tinnitus suppression system. As disclosed with reference to FIG. 13 below, a corresponding tinnitus suppression method is also part of the present disclosure.

The various modules or elements of the devices and systems disclosed herein can for instance be implemented in hardware, software or a combination thereof. For instance, the various modules and elements of the devices and systems disclosed herein may be implemented via application specific hardware components such as application specific integrated circuits, ASICs, and/or field programmable gate arrays, FPGAs, and/or similar components and/or application specific software modules being executed on multi-purpose data and signal processing equipment such as CPUs, DSPs and/or systems on a chip (SOCs) or similar components or any combination thereof.

For instance, the various modules or elements of the tinnitus suppression device discussed above may be implemented on a multi-purpose data and signal processing device configured for executing application specific software modules and for communicating with various sensor devices and/or neurostimulation devices or systems via conventional wireless communication interfaces such as an NFC, a WIFI and/or a Bluetooth interface.

Alternatively, the various modules or elements of the tinnitus suppression device and system discussed above may also be part of an integrated neurostimulation apparatus, further comprising specialized electronic circuitry (e.g., neurostimulation signal generators, amplifiers etc.) for generating and applying the multi-channel neurostimulation signal to a neurostimulation interface of the individual (e.g., a multi-contact spinal cord stimulation electrode, a deep brain stimulation (DBS) electrode, etc.).

The neurostimulation signals generated by the tinnitus suppression device described above may for instance also be transmitted to a neuronal stimulation device comprising a signal amplifier driving a multi-contact DBS electrode, spinal cord electrode, etc. that may already be implanted into a patient's nervous system for a purpose different than providing tinnitus suppression. Alternatively, dedicated DBS-like electrodes or spinal cord stimulation electrodes may be implanted for the purpose of applying the neurostimulation signals generated by the tinnitus suppression device via established and approved surgical procedures that were developed for implantation of conventional DBS electrodes or spinal cord stimulation electrodes etc. Further, as mentioned above the tinnitus suppression device may also be integrated together with a neuronal stimulation device into a single device.

### 4. SHORT DESCRIPTION OF THE FIGURES

Various aspects of the present disclosure are described in more detail in the following by reference to the accompanying figures. These figures show:

FIG. 1 a diagram illustrating an individual being equipped with a tinnitus suppression device according to aspects of the present disclosure;

FIG. 2 a functional block circuit diagram illustrating a tinnitus suppression device according to aspects of the present disclosure;

FIG. 3 a diagram illustrating how a tinnitus suppression device according to aspects of the present disclosure applies a filter operation to received sound signal generating three subcomponent signals that can be mapped to three different perceptual channels;

FIG. 4 a homunculus diagram illustrating how the three perceptual channels of FIG. 3 may be implemented via three different peripheral perceptive nerve fields;

FIG. 5 a diagram illustrating how a tinnitus suppression device as disclosed herein may operate in a multi-source outdoor auditory environment;

FIG. 6 a diagram illustrating the use of auxiliary sematic channels to improve sound perception facilitated by a tinnitus suppression device according to aspects of the present disclosure;

FIG. 7 a diagram illustrating how a tinnitus suppression device as disclosed herein can be used to support sound perception for individuals with residual hearing capabilities;

FIG. 8 a diagram illustrating how a tinnitus suppression device according to an embodiment of the present disclosure can be recalibrated in an automatic and on-line manner during operation;

FIG. 9 a diagram illustrating how the tinnitus suppression devices disclosed herein can be used to suppress, treat and/or ameliorate tinnitus;

FIG. 10 a diagram illustrating a tinnitus suppression device as disclosed herein in operation;

FIG. 11 a diagram illustrating a tinnitus suppression system as disclosed herein in operation;

FIG. 12 a diagram illustrating a tinnitus suppression method as disclosed herein;

FIG. 13 a diagram illustrating a tinnitus suppression method as disclosed herein;

### 5. DETAILED DESCRIPTION OF SOME EXEMPLARY ASPECTS

In the following, exemplary aspects of the present disclosure are described in more detail, with reference to a tinnitus suppression device that can be interfaced with neuronal stimulation electrodes such as spinal cord stimulation electrodes, DBS electrodes, etc., via an intermediate neuronal stimulation device. However, the present disclosure can also be used with any other neuronal stimulation interface that is capable of stimulating afferent sensory nerve fibers of the CNS targeting one or more sensory cortex areas of an individual.

While specific feature combinations are described in the following with respect to the exemplary aspects of the present disclosure, it is to be understood that not all features of the discussed aspects have to be present for realizing the technical advantages provided by the devices, systems, methods and computer programs provided by the present disclosure. The disclosed aspects and examples may be modified by combining certain features of one example aspect with one or more features of another aspect if technically feasible and functionally compatible. Specifically, the skilled person will understand that features, steps, components and / or functional elements of one example aspect can be combined with technically compatible features, steps, components and/or functional elements of any other example aspect. The present invention is defined by the appended claims.

Moreover, the various modules of the devices and systems disclosed herein can for instance be implemented in hardware, software, or a combination thereof. For instance, the various modules of the devices and systems disclosed herein may be implemented via application specific hardware components such as application specific integrated circuits, ASICs, and/or field programmable gate arrays, FPGAs, and/or similar components and/or application specific software modules being executed on multi-purpose data and signal processing equipment such as CPUs, DSPs and/or systems on a chip (SOCs) or similar components or any combination thereof.

For instance, the various modules of the tinnitus suppression device discussed herein above may be implemented on a multi-purpose data and signal processing device configured for executing application specific software modules and for communicating with various sensor devices and/or neurostimulation devices or systems via conventional wireless communication interfaces such as a Near Field Communication (NFC), a WIFI and/or a Bluetooth interface.

Alternatively, the various modules of the tinnitus suppression device provided by the present disclosure may also be part of an integrated neurostimulation apparatus, further comprising specialized electronic circuitry (e.g. neurostimulation signal generators, amplifiers etc.) for generating and applying the determined neurostimulation signals to a neurostimulation interface of the individual (e.g. a multi-contact electrode, a spinal cord stimulation electrode, a DBS electrode etc.).

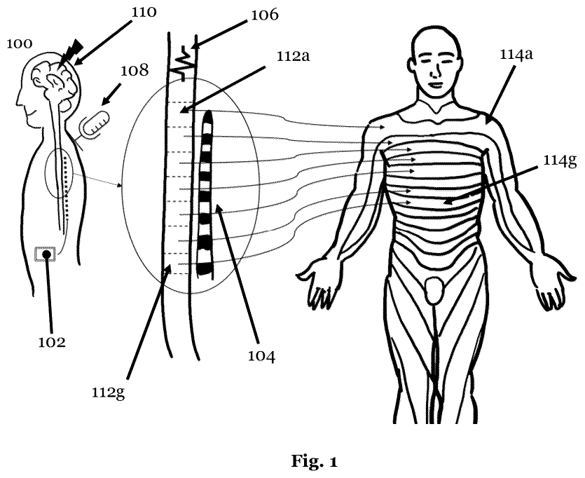

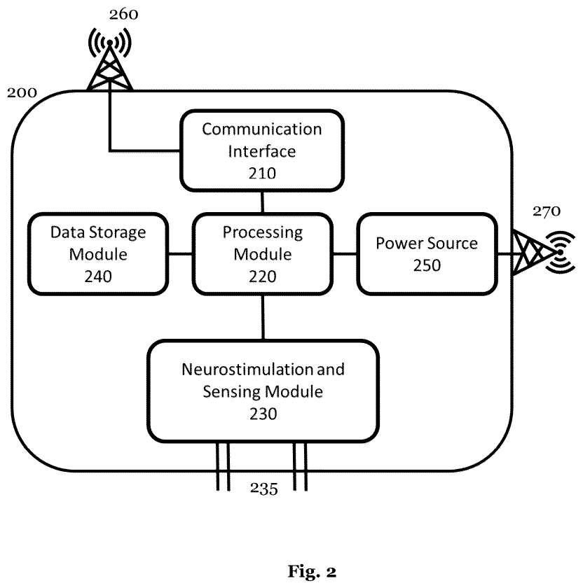

FIG. 1 illustrates a person/individual 100 that is equipped with a tinnitus suppression device as described in section 3 above and illustrated in an exemplary manner in FIG. 2 below. In the illustrated example, the tinnitus suppression device is implemented via direct neurostimulation of afferent sensory nerve fibers in the spinal cord via one or more multi-contact electrodes 104 driven by an implantable pulse generator (IPG) 102 that may be operatively/communicatively connected to or integrated with a tinnitus suppression device as disclosed herein.

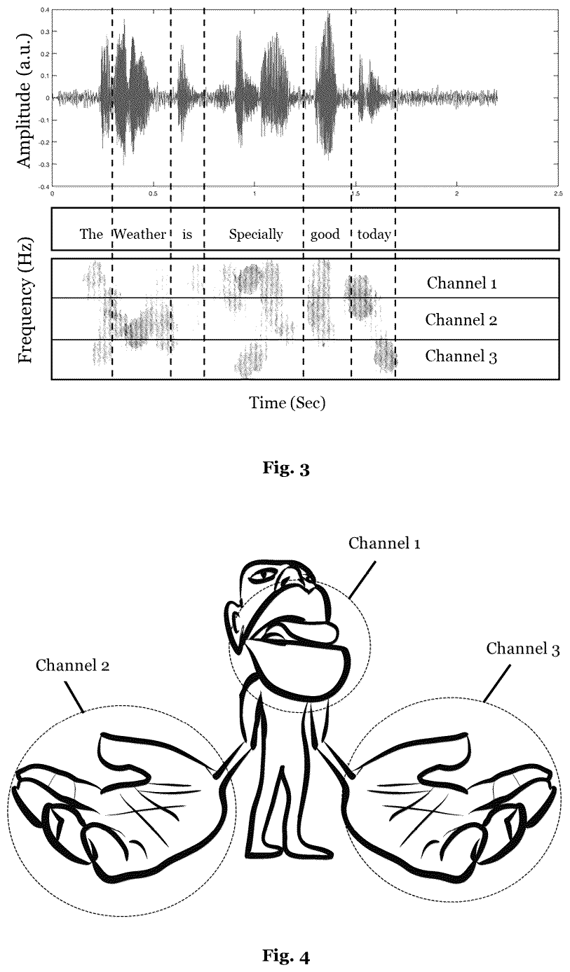

For establishing multiple perceptual communication channel to the brain of the individual 100 the tinnitus suppression device may be calibrated such that neurostimulation signals generated by the tinnitus suppression device and applied via the IPG 102 and the multi-contact electrode 104 elicit action potentials 106 in one or more afferent sensory nerve fibers of the spinal cord 106 targeting (e.g. via multi-synaptic afferent sensory pathways) one or more sensory cortex areas 110 of the individual 100 where the one or more action potentials 106 generate (directly or indirectly) artificial non-auditory sensory perceptions that can be used to represent a received sound signal (se FIG. 3 below) to be perceived by the brain of the individual 100. As discussed in detail in US 2020/0269049 A1, fully incorporated herein by reference, artificial sensory perceptions that are elicited in a sensory cortex area (e.g. a sensory cortex area processing touch sensations on the left or right hand) can also be associated with any kind of abstract information that is intelligible (i.e. consciously or subconsciously) by the individual 100.

In operation, the tinnitus suppression device receives sound signals recorded via one or more sound sensors/microphones 108 that may be worn by the individual 100, be integrated with the tinnitus suppression device and/or be provided by a general-purpose data and signal processing device such as a smart phone. For instance, some or all functionalities of the tinnitus suppression devices discussed in detail in section 3 above, may be implemented via application specific software modules executed by such a general-purpose data and signal processing device which in turn may be interfaced (e.g., wirelessly) with the IPG 102 or a similar neurostimulation device operating in conjunction to implement aspects of the tinnitus suppression device disclosed herein.

For the embodiment illustrated in FIG. 1 the perceptual channels correspond to different dermatomes 114a-114g innervated by spinal nerve fibers branching of the spinal cord at location 112a to 112g. In this general example different contacts of the stimulation electrode may be used to stimulate regions of the spinal cord typically relaying sensory information from a given dermatome (e.g., a dermatome 114a located on the front torso of the person).

In other aspects, complex, multi-contact neural stimulation signals may also be used to selectively stimulate single peripheral nerve fields within a given dermatome or combinations of dermatomes and/or peripheral nerve fields.

FIG. 2 shows an exemplary tinnitus suppression device 200 according to aspects of the present disclosure. The exemplary tinnitus suppression device 200 comprises an integrated neurostimulation and sensing module 230 (e.g. comprising a neuronal signal generator and an output amplifier as well as a sensing amplifier and an analog to digital converter and similar circuitry) that is connected to a plurality of output signal leads 235 and a plurality of separate or identical sensing signal leads 235 that may be interfaced with a neurostimulation interface of the individual (e.g. a multi-contact spinal cord stimulation electrode such as the electrode 104 shown in FIG. 1). In other aspects the neurostimulation and sensing module 230 may be replaced by a simpler neurostimulation module that is not configured for sensing neural bioelectric activity or signals.

The exemplary tinnitus suppression device 200 may further comprise a communication antenna 260 operably connected to a communication interface module 210, configured for wireless communication (e.g., via NFC, Bluetooth, or a similar wireless communication technology). The communication interface module 210 may be configured, for example, to receive one or more sound signals from one or more sound sensors (not shown; e.g., a set of microphones worn by the individual) and/or control information from a control device such as a remote control or a smart phone. The communication interface module 210 may also function as a user interface configured to receive user inputs.

The communication interface module 210 is operably connected to a data/signal processing module 220 configured to generate one or more neurostimulation signals and/or signal parameters (e.g., waveform, pulse shape, amplitude, frequency, burst count, burst duration etc.) for generating the one or more neurostimulation signals. For instance, the processing module 220 may access a data storage module 240 configured to store a plurality of sound signal filters for the various filter modes as described in section 3. above and/or relations, specific for the individual, associating a plurality of neurostimulation signals (or parameters used for generating a plurality of neurostimulation signals) with a plurality of corresponding pieces of auxiliary information to be communicated to the individual, e.g., for establishing a perceptual channel used to indicate the sound source direction, the motional state of the sound signal source and/or context information such as the emotional state of a speaker.

The generated neurostimulation signals and/or the signal parameters are input into the integrated neurostimulation and sensing module 230 that may be configured to process (e.g., modulate, switch, amplify, covert, rectify, multiplex, phase shift, etc.) the one or more (multi-channel) neurostimulation signals generated by the processing module 220 or to generate the one or more neurostimulation signals based on the signal parameters provided by the processing module 220.

The generated and processed neurostimulation signals are then output by the neurostimulation and sensing module 230 and can be applied to one or more electric contacts of a neurostimulation electrode (e.g., a DBS electrode or spinal cord stimulation electrode as shown in FIG. 1) via output leads 235. The tinnitus suppression device of FIG. 2 may also comprise a rechargeable power source 250 that, for instance may be wirelessly charged via a wireless charging interface 265.

As discussed in section 3. above, the processing module 220 and the neurostimulation module 230 may be configured to determine a received sound signal that corresponds to a template stored in the memory 240, that is above a preconfigured threshold, preferably above a preconfigured threshold which is specific for the individual, or both, and generate a multi-channel neurostimulation signal encoding the determined sound signal, and apply the generated multi-channel neurostimulation signal to a neurostimulation device of the individual configured to directly stimulate afferent sensory neurons of the central nervous system, CNS, of the individual and thereby to elicit, for each channel of the neurostimulation signal, one or more non-auditory, preferably somatosensory, perceptions in a cortex area of the individual.

As discussed above, the data/signal processing module 220 may be further configured to, e.g., in conjunction with the data storage module 240 and the neurostimulation and sensing module 230, to execute an on-line autocalibration method as discussed in section 3 above. Further, the tinnitus suppression device may also comprise a transmitter module (e.g., the communication interface 210) as an alternative to the neurostimulation and sensing module 230 to communicate with a remote neurostimulation device.

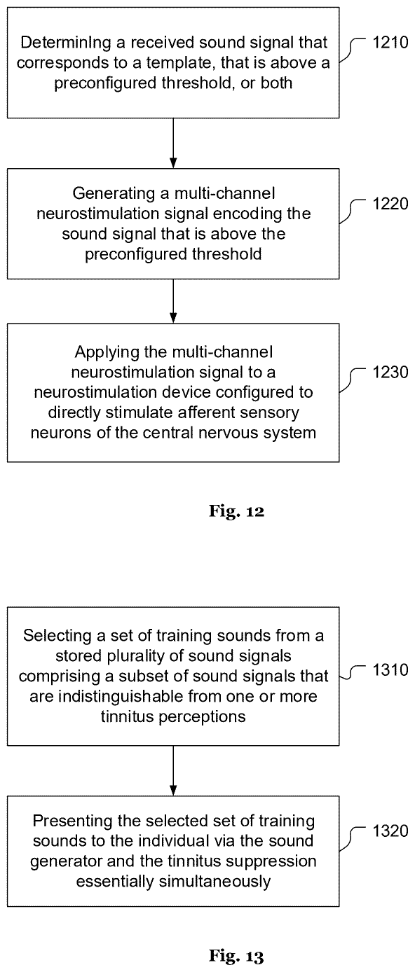

The processing module 220 and the neurostimulation module 230 may be further configured to carry out the various steps discussed in section 3. above and disclosed with reference to the method of FIG. 12 below.

FIG. 3 and FIG. 4 illustrate a general example how some aspects of the present disclosure can be used to establish a three-channel, non-auditory hearing aid and tinnitus suppression device for a patient. Specifically, the processing module filters a received sound signal (see waveform in top trace of FIG. 3) via a three-channel filter bank (see spectrogram in lower trace of FIG. 3).

The output signal of each bandpass filter of the filter bank (i.e., a subcomponent signal as discussed in detail in section 3 above) is then separately sampled and used to generate a three-channel neurostimulation signal. As shown in the homunculus diagram of FIG. 4 each of the subcomponent signals is configured to elicit an artificial sensation perceived by the individual in the lips (channel 1; high frequency components of the received sound signal), in the right hand (channel 2, medium frequency components of the received sound signal) and the left hand (channel 3, low frequency components of the received sound signal).

As discussed in detail in section 3 above, instead of a filter bank, other filter operations such as wavelet or Gabor filters may also be used to subdivide a received sound signal into subcomponent signals that are then mapped to different perceptual channels.

In some embodiments, the disclosed tinnitus suppression device may be calibrated and N perceptual channels are identified as discussed in section 3 above. Each different channel could then be mapped to a different frequency band. The number N (and the differentiated levels within each channel) will define the maximum resolution or bandwidth of the perceptual/transmission matrix, which relate to a specific characteristic of the implant type and implant location with respect to the neural tissue defined per individual patient. The decomposition algorithm/filter operation of sound signals can be customized, so that e.g., an ICA is conducted which solves for a target number of components equals N. This decomposition matrix may be fixed for the patient and subsequently a completely customized translation of the sound signal occurs that is optimized for the respective patient. In some embodiments, here, pre-calculated ICA decomposition matrices may be applied which are based on e.g. language-specific audio file training sets.

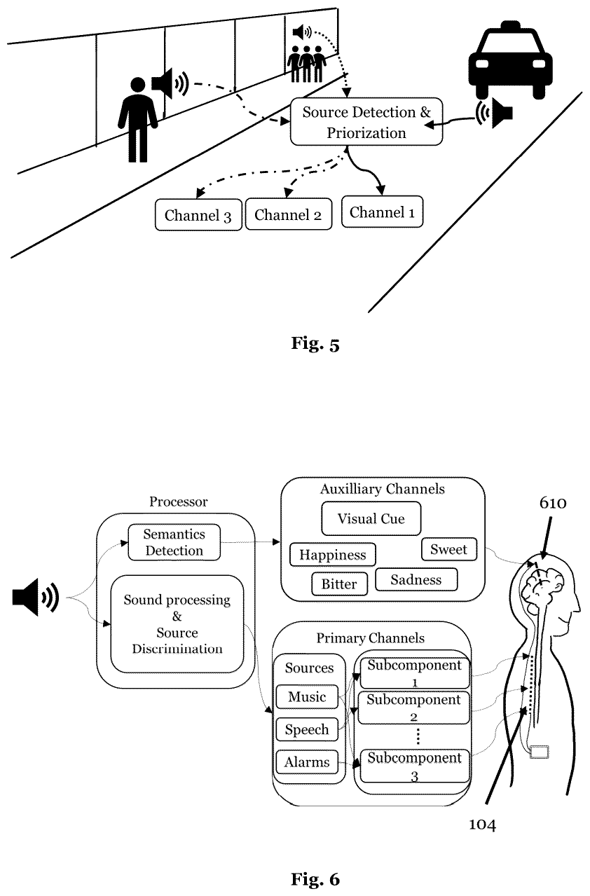

FIG. 5 illustrates how some embodiments of the disclosed tinnitus suppression device 200 can be equipped with source detection/discrimination modules (soft-and/or hardware based) that can enable the tinnitus suppression device 200 to determine which part of a complex auditory environment should be perceived by the individual (not shown) with high fidelity and/or priority (e.g., the sound of an approaching car), which sounds with low fidelity/priority (e.g., a person 520 directly talking to the individual) and which sounds are to be filtered out completely (e.g., background noise generated by a remote group of people 530 talking).

As discussed in section 3 above, the filter modes and/or filter function stored in the memory module 240 of the tinnitus suppression devices 200, can, for example, automatically be selected by the processing module, after a determination that the individual is located in an outdoor environment with likelihood of motorized traffic. A traffic filter mode may for example use a specialized spatio-temporal filter operation to filter out sounds typically generated by dangerous objects (e.g., cars) with high fidelity and select one of the perceptual channels to transmit this subcomponent signal with high priority and/or signal strength.

FIG. 6 illustrates an embodiment of the disclosed tinnitus suppression devices that is configured to transmit auxiliary information such as a sound signal duration or context information such as the emotional state of a speaker via a separate DBS electrode 610, while at the same time an SCS-electrode 104 (as illustrated in detail in FIG. 1 above) is operated to transmit the multi-channel neurostimulation signal used for sound signal representation. As discussed above, the processing module of the tinnitus suppression device is configured to map, based on a selected filter mode and/or operation different types of sound signal sources (music, speech, alarms) to different perceptual channel addressable via the SCS-electrode. In addition to the source discrimination and prioritization module discussed for FIG. 5 above, the processor may also comprise or execute a semantics and/or context detection module that allows the tinnitus suppression device to determine relevant context information, such as the language used by a sound source.

For instance, an auxiliary taste channel may be used to signal to the individual whether a sound signal source uses a foreign language (sweet) or the native language of the individual. In another example the emotional state may be encoded as artificial taste sensations, e.g. (aggressive=bitter; empathic=sweet). For instance, modern speech processing software (e.g., trained multi-layered neural networks) may be used automatically extract meaning and/or context of received speech signals.

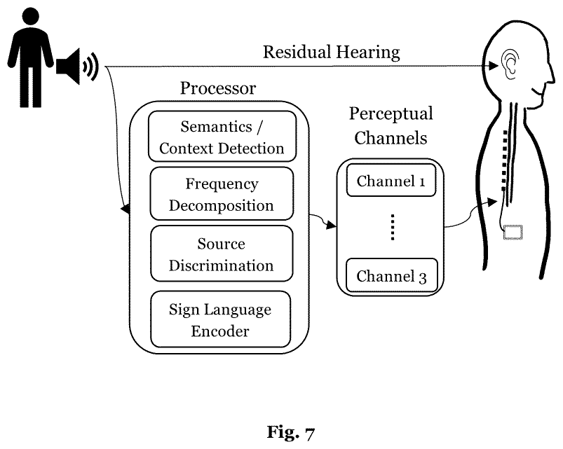

FIG. 7 illustrates that some embodiments of the present disclosure can also be used to supplement or support persons having residual hearing providing even further benefits over conventional technologies. FIG. 7 also illustrates, that in some embodiments, the tinnitus suppression device may also comprise a hard-and/or software implemented sign language encoder module that can support sound perception by the individual by operating in a sign-language assistance mode. For example, all or part of the typical sign-language hand poses can be translated into a combination of individually detectable perceptual channels and be used to support sound perception by the individual.

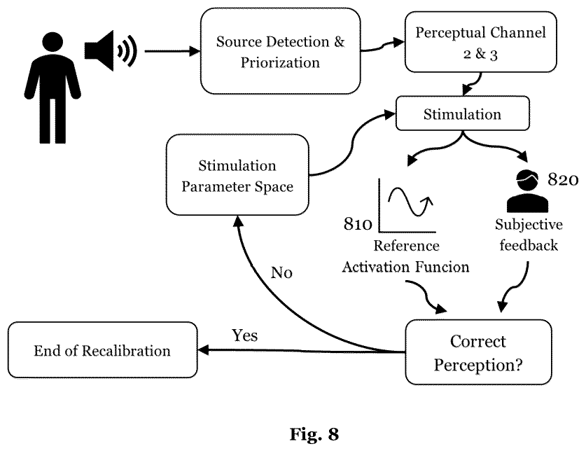

FIG. 8 illustrates the auto-recalibration procedure that is discussed in detail in section 3 above. For instance, in some aspects, while the disclosed tinnitus suppression device receives sound signals and processes (e.g., filters, maps, etc.) them as discussed above the neuronal sensing module 230 (see FIG. 2 above) constantly records the bioelectric responses (e.g., ECAP or somatosensory EESP, or extracellularly measured action potentials or similar bioelectric response) of the stimulated nerves/nerve fibers/neurons and derives an activation function that can be compared to a reference activation function 810 (as disclosed in US patent application Ser. No. 17/224,953, incorporated herein in it's entirety). Alternatively or preferably additionally, sensory feedback 820 from the patient can be used to determine whether the fidelity of the sound signal representation is still optimal or may be improved by readjusting the signal parameters and/or the filter operation used to generate the multi-channel neurostimulation signal. In this manner, the performance of the non-auditory hearing aid implemented by the tinnitus suppression device can be maintained as good as possible even in normally behaving (e.g., moving) patients.

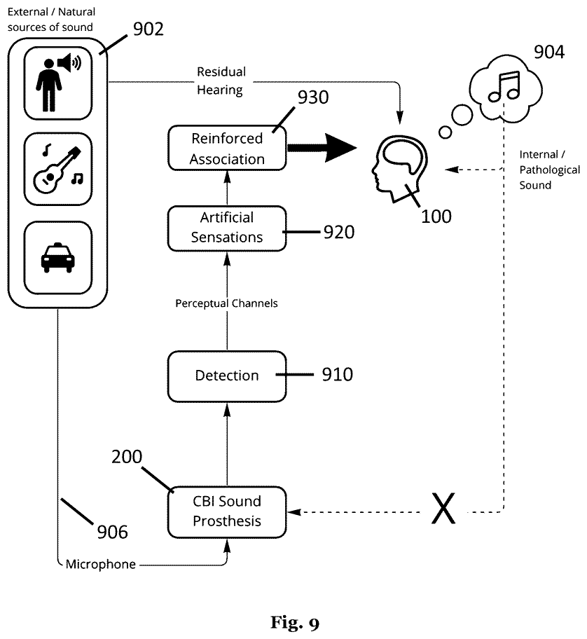

FIG. 9 illustrates a tinnitus suppression device 200 as disclosed herein in operation to suppress hallucinatory/tinnitus related sensations 904 of an individual 100. The tinnitus suppression device 200 (also designated CBI sound prosthesis herein) receives real sound signals 902 via a microphone 906 and a receiver module as disclosed herein. As discussed in section 3 above, the tinnitus suppression device 200 is configured to detect/determine 910 when a received sound signal 902 corresponds to a template stored in the memory 240, when it is above a preconfigured threshold, preferably above a preconfigured threshold which is specific for the individual 100, or both, and in response generate a multi-channel neurostimulation signal encoding the determined sound signal, and apply the generated multi-channel neurostimulation signal to a neurostimulation device of the individual to elicit artificial sensations that the individual associates with the real sound signal 902. Since the tinnitus suppression device 200 does not generate non-auditory sound perceptions for hallucinatory/tinnitus related sensations 904 real sounds 902 get reinforced 930 as compared to for hallucinatory/tinnitus related sensations 904 which enables the brain of the individual 100 to discriminate between hallucinatory/tinnitus related sensations 904 and real sounds 902 and thereby to suppress tinnitus. For instance, the memory of the tinnitus suppression device may store a library of sounds known to be triggering tinnitus in the individual or otherwise be associated with a tinnitus state or perception of the individual.

As also disclosed elsewhere herein, the tinnitus suppression device 200 may also employ auxiliary perceptual channels to tag/flag real sounds 902 transmitted by the multi-channel neurostimulation signal that are substantially indistinguishable from the hallucinatory/tinnitus related sensations 904 and thereby improve tinnitus suppression. Further, the tinnitus suppression device 200 may further be configured to carry out one or more of the steps disclosed in section 3. above which are not repeated here for conciseness.

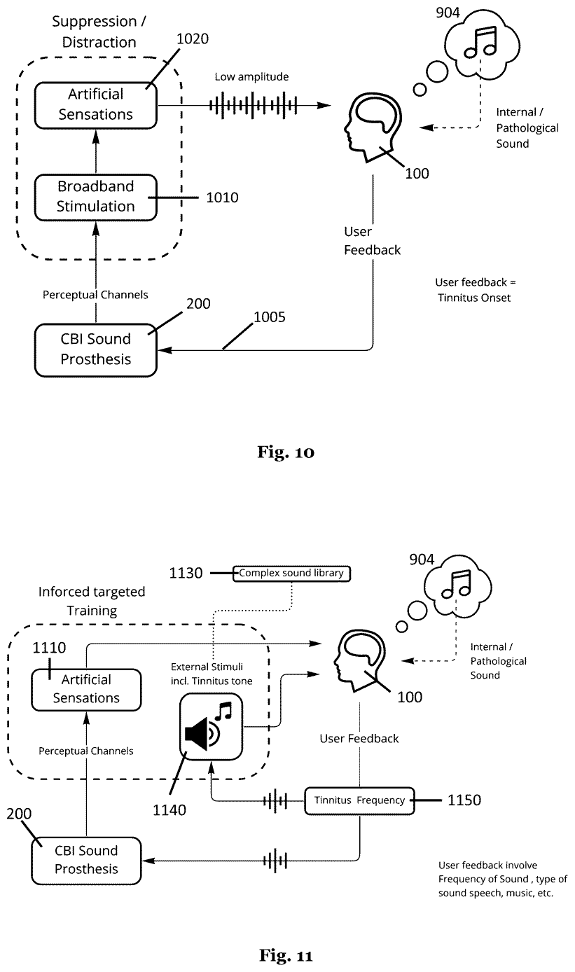

FIG. 10 illustrates a tinnitus suppression device 200 according to some aspects of the present disclosure in operation to suppress hallucinatory/tinnitus related sensations 904 of an individual 100. The illustrated exemplary tinnitus suppression device 200 (also designated CBI sound prosthesis herein) comprises a user input interface operably connected to the processing module and configured to receive a user input 1005 indicating a tinnitus state of the individual. In response to the user input indicating the tinnitus state of the individual, the processing module and the neurostimulation module of the tinnitus suppression device 200 may generate a multi-channel neurostimulation signal encoding a non-auditory perception configured to suppress the tinnitus state of the individual such as a broadband noise signal 1010 having low amplitude. The broadband noise signal 1010 may be applied to a neurostimulation device of the individual and be configured to elicit a non-auditory sound perception 1020 corresponding to a white noise sound or a similar noise signal as discussed in section 3 above, depending on the power spectral density of the multi-channel stimulation signal.

In some aspects the user input 1005 may indicate one or more of: an onset of a tinnitus perception of the individual 100, a type of tinnitus perception, a frequency of tinnitus perception, or an intensity of tinnitus perception.

In other aspects, the processing module and the neurostimulation module of the tinnitus suppression device 200 may be further configured to determine that a received sound level is, for a preconfigured duration, below a second preconfigured threshold, preferably below a second preconfigured threshold that is specific for the individual, and in response generate, based on the determination, and optionally based on stored parameters characterizing a tinnitus perception (e.g., based on the user input 1005) the multi-channel neurostimulation signal encoding a non-auditory perception configured to suppress a tinnitus state of the individual.

FIG. 11 illustrates a tinnitus suppression system as disclosed herein in operation. In some aspects, the system includes a the tinnitus suppression device as disclosed herein, a sound generator 1140 comprising a loud speaker, a memory module 1130 storing a plurality of training sounds comprising a subset of sound signals that are effectively indistinguishable from one or more tinnitus perceptions of an individual 100. The illustrated system further comprises a training module (e.g., integrated into the sound generator 1140) which is operably connected to the tinnitus suppression device 200, the memory 1130 and the sound generator 1140 and configured to select a set of training sounds from the stored plurality of sound signals comprising a subset of sound signals that are indistinguishable from one or more tinnitus perceptions of individual and present the selected set of training sounds to the individual via the sound generator 1140 and the tinnitus suppression device 200 essentially simultaneously, e.g., such that the non-auditory sound perception 1110 is perceived by the brain of the individual 100 effectively simultaneously with hearing the sound generated by the sound generator 1140.

In some aspects, the tinnitus suppression system may further comprise an input interface operably connected to the memory and configured to receive parameters characterizing one or more tinnitus perceptions of the individual. The training module may further be configured to generate, based on the received parameters, a first set of training sounds that are indistinguishable from one or more tinnitus perceptions of the individual and a second set of training sounds that are distinguishable from one or more tinnitus perceptions of the individual and store both sets in the memory 1130 for use during training the individual to suppress perceiving tinnitus.

The system can also provide feedback to the individual not only when a defined sound (tinnitus triggering) frequency or sound type is detected via microphone in the real world. The system can additionally inform the individual if the defined external stimuli is not present. For example, a low frequency short bursts of stimulus may indicate when the external sound is not present then switching to normal operation mode when the external sound is detected.

FIG. 12 shows a tinnitus suppression method according to aspects of the present disclosure. For instance, the method may be carried out by a tinnitus suppression device as disclosed herein, e.g., when executing a computer program as disclosed in section 3. above. For example, the tinnitus suppression device (cf. FIG. 2 for example) may comprise a receiver module configured to receive sound signals, a processing module operably connected to a memory and to the receiver module and a neurostimulation module operably connected to the processing module.

At step 1210 a received sound signal is determined that corresponds to a template, that is above a preconfigured threshold, preferably above a preconfigured threshold which is specific for the individual, or both. At step 1220 a multi-channel neurostimulation signal is generated that encodes the determined sound signal and at step 1230 the generated multi-channel neurostimulation signal is applied to a neurostimulation device of the individual configured to directly stimulate afferent sensory neurons of the central nervous system, CNS, of the individual and thereby to elicit, for each channel of the neurostimulation signal, one or more non-auditory, preferably somatosensory, perceptions in a cortex area of the individual.

In some aspects, the memory stores parameters characterizing a tinnitus perception of the individual and the method further comprises comparing the received sound signal to the stored parameters characterizing a tinnitus perception of the individual and determining, based on the comparison, whether an auditory perception of the received sound signal is distinguishable from the tinnitus perception of the individual, and signaling to the individual, via the neurostimulation device, whether the multi-channel neurostimulation signal corresponds to a sound signal that is distinguishable from the tinnitus perception of the individual or not.

Further aspects of the tinnitus suppression method are disclosed herein in section 3. with reference to a tinnitus suppression device and are not repeated here for conciseness.

FIG. 13 shows a tinnitus suppression method according to aspects of the present disclosure. For instance, the method may be carried out by a tinnitus suppression system as disclosed herein, e.g., when executing a computer program as disclosed in section 3. above. For example, the tinnitus suppression system (cf. FIG. 11 for example) may comprise a tinnitus suppression device as disclosed herein, a sound generator comprising a loudspeaker, a memory storing a plurality of training sounds comprising a subset of sound signals that are indistinguishable from one or more tinnitus perceptions of an individual and a training module operably connected to the tinnitus suppression device, the memory and the sound generator.

At step 1310 a set of training sounds is selected from the stored plurality of sound signals comprising a subset of sound signals that are indistinguishable from one or more tinnitus perceptions of the individual.

At step 1310 the selected set of training sounds is presented to the individual via the sound generator and the tinnitus suppression device essentially simultaneously.

In some aspects, the tinnitus suppression system may comprise an input interface operably connected to the memory and the method may further comprise receiving parameters characterizing one or more tinnitus perceptions of the individual and generating based on the received parameters, a first set of training sounds that are indistinguishable from one or more tinnitus perceptions of the individual and a second set of training sounds that are distinguishable from one or more tinnitus perceptions of the individual and store both sets of training sounds in the memory.

Further aspects of the tinnitus suppression method are disclosed herein in section 3. with reference to a tinnitus suppression system and are not repeated here for conciseness.

1. A tinnitus suppression device for an individual, comprising:
a receiver configured to receive sound signals;
a processor operably connected to a memory and to the receiver; and
a stimulator operably connected to the processor; wherein the processor and the stimulator are configured to:
determine a received sound signal that corresponds to a template stored in the memory, that is above a preconfigured threshold which is specific for the individual, or both;
generate a multi-channel neurostimulation signal encoding the determined sound signal; and
apply the generated multi-channel neurostimulation signal to a neurostimulation device of the individual configured to directly stimulate afferent sensory neurons of the central nervous system, CNS, of the individual and thereby to elicit, for each channel of the neurostimulation signal, one or more non-auditory perceptions in a cortex area of the individual.

2. The tinnitus suppression device of claim 1, wherein the memory stores parameters characterizing a tinnitus perception of the individual; and
wherein the processor and the stimulator are further configured to:
compare the received sound signal to the stored parameters characterizing a tinnitus perception of the individual;
determine, based on the comparison, whether an auditory perception of the received sound signal is distinguishable from the tinnitus perception of the individual; and
signaling to the individual, via the neurostimulation device, whether the multi-channel neurostimulation signal corresponds to a sound signal that is distinguishable from the tinnitus perception of the individual or not.

3. The tinnitus suppression device of claim 1, further comprising:
a user input interface operably connected to the processor and configured to receive a user input indicating a tinnitus state of the individual; wherein, in response to the user input indicating the tinnitus state of the individual, the processor and the stimulator are further configured to:
generate a second multi-channel neurostimulation signal encoding a non-auditory perception configured to suppress the tinnitus state of the individual; and
apply the generated second multi-channel neurostimulation signal to the neurostimulation device of the individual.

4. The tinnitus suppression device of claim 1, wherein the processor and the stimulator are further configured to:
determine that a received sound level is, for a preconfigured duration, below a second preconfigured threshold that is specific for the individual;
generate, based on the determination that the received sound level is below the second preconfigured threshold for the preconfigured duration, a second multi-channel neurostimulation signal encoding a non-auditory perception configured to suppress a tinnitus state of the individual; and
apply the generated second multi-channel neurostimulation signal to the neurostimulation device of the individual.

5. The tinnitus suppression device of claim 4, wherein the second multi-channel neurostimulation signal encoding the non-auditory perception configured to suppress the tinnitus state of the individual comprises a multi-channel neurostimulation signal encoding a noise signal having a power spectrum corresponding to white noise, pink noise, grey noise, blue noise, violet noise or Brownian noise.

6. The tinnitus suppression device of claim 5, wherein the processor and the stimulator are further configured to randomly cycle between different multi-channel neurostimulation signals each encoding a different noise signal; or wherein the processor and the stimulator are further configured to generate bursts of the multi-channel neurostimulation signal encoding a specific noise signal such that there are rhythmic active and silent segments delivered to the individual.

7. The tinnitus suppression device of claim 1, wherein encoding by the processor comprises:
applying a filter operation to the received sound signal to generate a plurality of subcomponent signals of the sound signal; and
mapping each subcomponent signal to a different channel of the multi-channel neurostimulation signal.

8. The tinnitus suppression device of claim 1, wherein the processor is configured to
determine, via an on-line auto-calibration procedure, a maximal number of different perceivable perceptual channels that are specific for the individual; and
select the applied filter operation based on the determination, such that a fidelity of a representation of the received sound signal by the plurality of subcomponent signals is maximized for the determined number of channels.

9. The tinnitus suppression device of claim 8,
wherein the processor is further configured to apply the filter operation according to multiple selectable filter modes, and
wherein the generation of the subcomponent signals and/or the mapping of the subcomponent signals to the multiple channels of the neurostimulation signal is based on the selected filter mode.

10. The tinnitus suppression device of claim 1, wherein the processor is further configured to:
determine, based on an analysis of the received sound signal, one or both of an auditory environment and a likely type of sound signal source associated with the received sound signal; and
encode the received sound signal based on one or both of the determined auditory environment and type of sound signal source.

11. A tinnitus suppression system, comprising:
a tinnitus suppression device, comprising:
a receiver configured to receive sound signals;
a processor operably connected to a memory and to the receiver; and
a stimulator operably connected to the processor; wherein the processor and the stimulator are configured to:
determine a received sound signal that corresponds to a template stored in the memory, that is above a preconfigured threshold which is specific for the individual, or both;
generate a multi-channel neurostimulation signal encoding the determined sound signal; and
apply the generated multi-channel neurostimulation signal to a neurostimulation device of the individual configured to directly stimulate afferent sensory neurons of the central nervous system, CNS, of the individual and thereby to elicit, for each channel of the neurostimulation signal, one or more non-auditory perceptions in a cortex area of the individual
a sound generator comprising a loud speaker;
wherein the memory stores a plurality of training sounds comprising a subset of sound signals that are indistinguishable from one or more tinnitus perceptions of an individual;
a training module operably connected to the tinnitus suppression device, the memory and the sound generator and configured to:
select a set of training sounds from the stored plurality of sound signals comprising a subset of sound signals that are indistinguishable from one or more tinnitus perceptions of individual; and
present the selected set of training sounds to the individual via the sound generator and the tinnitus suppression device essentially simultaneously.

12. The tinnitus suppression system of claim 11, further comprising:
an input interface operably connected to the memory and configured to receive parameters characterizing one or more tinnitus perceptions of the individual; and wherein the training module is configured to generate, based on the received parameters, a first set of training sounds that are indistinguishable from one or more tinnitus perceptions of the individual and a second set of training sounds that are distinguishable from one or more tinnitus perceptions of the individual.

13. A non-transitory computer-readable memory medium storing program instructions which, when executed by a processor, cause a tinnitus suppression device to:
determine a received sound signal that corresponds to a template, that is above a preconfigured threshold which is specific for the individual, or both;
generate a multi-channel neurostimulation signal encoding the determined sound signal; and
apply the generated multi-channel neurostimulation signal to a neurostimulation device of the individual configured to directly stimulate afferent sensory neurons of the central nervous system (CNS) of the individual and thereby to elicit, for each channel of the neurostimulation signal, one or more non-auditory perceptions in a cortex area of an individual.

14. The non-transitory computer-readable memory medium of claim 13, wherein the program instructions are further executable to cause the tinnitus suppression device to:
select a set of training sounds from a stored plurality of sound signals comprising a subset of sound signals that are indistinguishable from one or more tinnitus perceptions of an individual; and
present the selected set of training sounds to the individual via a sound generator and the tinnitus suppression device substantially simultaneously.

15. The non-transitory computer-readable memory medium of claim 13,
wherein the memory medium stores parameters characterizing a tinnitus perception of the individual; and wherein the program instructions are further executable by the processor to cause the tinnitus suppression device to:
compare the received sound signal to the stored parameters characterizing a tinnitus perception of the individual;
determine, based on the comparison, whether an auditory perception of the received sound signal is distinguishable from the tinnitus perception of the individual; and
signaling to the individual, via the neurostimulation device, whether the multi-channel neurostimulation signal corresponds to a sound signal that is distinguishable from the tinnitus perception of the individual or not.

16. The non-transitory computer-readable memory medium of claim 13,
wherein, in response to user input indicating a tinnitus state of the individual received by a user input interface, the program instructions are further executable by the processor to cause the tinnitus suppression device to:
generate a second multi-channel neurostimulation signal encoding a non-auditory perception configured to suppress the tinnitus state of the individual; and
apply the generated second multi-channel neurostimulation signal to the neurostimulation device of the individual.

17. The non-transitory computer-readable memory medium of claim 13, the program instructions are further executable by the processor to cause the tinnitus suppression device to:
determine that a received sound level is, for a preconfigured duration, below a second preconfigured threshold that is specific for the individual;
generate, based on the determination that the received sound level is below the second preconfigured threshold for the preconfigured duration, a second multi-channel neurostimulation signal encoding a non-auditory perception configured to suppress a tinnitus state of the individual; and
apply the generated second multi-channel neurostimulation signal to the neurostimulation device of the individual.

18. The non-transitory computer-readable memory medium of claim 13, wherein the second multi-channel neurostimulation signal encoding the non-auditory perception configured to suppress the tinnitus state of the individual comprises a multi-channel neurostimulation signal encoding a noise signal having a power spectrum corresponding to white noise, pink noise, grey noise, blue noise, violet noise or Brownian noise.

19. The non-transitory computer-readable memory medium of claim 13, wherein the program instructions are further executable by the processor to cause the tinnitus suppression device to:
randomly cycle between different multi-channel neurostimulation signals each encoding a different noise signal; or
generate bursts of the multi-channel neurostimulation signal encoding a specific noise signal such that there are rhythmic active and silent segments delivered to the individual.

20. The non-transitory computer-readable memory medium of claim 13, wherein the program instructions are further executable by the processor to encode the determined sound signal by:
applying a filter operation to the received sound signal to generate a plurality of subcomponent signals of the sound signal; and
mapping each subcomponent signal to a different channel of the multi-channel neurostimulation signal.
